(19) United States
(12) Patent Application Publication
(10) Pub. No.: US 2006/0089642 A1
(43) Pub. Date: Apr. 27, 2006

(54) Prefracture spinal implant for osteoporotic unfractured bone

(76) Inventors: Robert L. Diaz, Palm Beach Gardens, FL (US); David R. Campbell, Jupiter, FL (US)

Correspondence Address: MCHALE & SLAVIN, P.A., 2855 PGA BLVD, PALM BEACH GARDENS, FL, 33410, US

(21) Appl. No.: 10/976,192

(22) Filed: Oct. 27, 2004

(51) Int. Cl. A61B 17/68; A61F 2/28; A61B 17/70

(52) U.S. Cl. 606/60; 623/23.44; 623/23.49; 606/61

(57) ABSTRACT

An implant for vertebrae or other bones, either fractured or at risk of fracture, includes a load bearing support member carrying a bone growth material to increase bone density and long term treatment.

### RELATED APPLICATIONS

This application is related to U.S. patent application Ser. No. 10/838523 filed May 3, 2004 and U.S. patent application Ser. No. 10/______ (Express Mail No. EV470270283 May 3, 2004).

### FIELD OF THE INVENTION

The instant invention relates generally to devices useful for the prevention of fractures in bones and the repair of fractured bones with minimum trauma; particularly to the prevention of fractures in bones which are at increased risk for fracture and most particularly to administration of spinal implants serving as a structural carrier for the administration of and slow release of one or more bone growth enhancing agents to the interior surfaces of a vertebral body to enhance bone growth, resulting in increased bone density, strength and mechanical resistance to vertebral body compression fracture, thus reducing susceptibility of the vertebral body to fracture.

### BACKGROUND OF THE INVENTION

Bones provide an organism support and protection, for example, support for muscle movement and protection for organs. Living bone tissue is in a constant state of flux due to the process of bone remodeling. In the process of bone remodeling, the mineralized bone matrix is continuously deposited and resorbed. Bone cells termed “osteoclasts” and “osteoblasts” carry out bone remodeling. Osteoclasts remove tissue from the bone surface and osteoblasts replace this tissue.

Rapid turnover of bone occurs throughout childhood as bones increase in size and thickness until the individual reaches a genetically-determined adult height. At adult height bones cease to grow in size but continue to increase in thickness until the individual reaches approximately 30 years of age. As bone growth ceases, the activity of osteoblasts and osteoclasts becomes imbalanced and bone is resorbed faster than it is replaced, thus leading to a gradual thinning of the bones. With thinning the microarchitexture of bone tissue deteriorates creating spaces or pores between the normally dense units of the bony matrix.

“Porous bone”, a pathological condition termed “osteoporosis” occurs with chronic thinning of bones. The hallmark of osteoporosis is increased fragility of bones due to the loss of bone from the interior of the medullary canal. Such bone loss reduces the overall density of bone tissue (osteopenia). As a bone thins it becomes increasingly susceptible to fracture with minimum trauma.

The vertebral column, also referred to as “spine” or “backbone”, is especially prone to fracture as it forms a major load-bearing structure of the body. The vertebral column comprises 7 cervical vertebrae (neck), 12 thoracic vertebrae (chest/ribs); 5 lumbar vertebrae (lower back); 1 sacrum (fusion of 5 sacral vertebrae) and 1 coccyx (referred to as “tailbone”, fusion of 4 coccygeal vertebrae). When a vertebral body fractures, it collapses, tilting the spine forward and reducing it's overall length, thus the posture of the osteoporotic patient suffering from vertebral body fractures (VCFs) becomes hunched over (kyphotic) with an accompanying reduction in height of the patient. The osteoporotic patient with spine fractures experiences decreased mobility leading to an inability to carry out everyday tasks and thus suffers an overall reduction in quality of life. Untreated, these vertebral body fractures lead to further fracturing, progressive spinal deformity and misalignment, disturbance and deformity of the intervertebral disks and chronic pain from the dysfunction of muscles, tendons and ligaments by the misshapen spine. Additionally, further health problems may result due to the compression of internal organs by the misaligned spine including malnutrition, falls with hip and wrist fractures and other problems.

Ideally, therapeutic measures for thinning bone should add support structure and restore bone density and thus reduce susceptibility to additional fractures. Preventing fracture of osteoporotic bone significantly improves the health, well-being and functional capabilities of the osteoporotic patient.

Other bone-related diseases and/or defects may involve thinning of the bones, for example, after a traumatic injury to a limb with resultant disuse osteopenia, corticosteroid regimens, complications with prosthetic devices and damage due to radiation treatments.

Although there is much information in the art regarding factors and methods which can influence bone remodeling, information is more limited on factors and methods which can directly stimulate bone growth in general. What is needed in the art is an efficient method which can achieve enhanced bone growth in areas specifically affected by osteopenia, thus increasing bone density in these affected areas and reducing susceptibility of the thinning bones to fracture.

### DESCRIPTION OF THE PRIOR ART

Numerous and varied treatments for osteoporosis can be found in the prior art; a few examples of such treatments follow.

U.S. Pat. Nos. 4,904,478 and 5,228,445 disclose the use of a slow release sodium fluoride preparation which when administered maintains a safe and effective serum level of fluoride useful for the treatment of osteoporosis. This preparation stimulates bone formation and improves bone quality thus aiding in the prevention of bone fractures which are often a frequent occurrence in osteoporetic patients.

U.S. Pat. No. 5,614,496 discloses a method for administration of FGF-1 in order to promote bone repair and growth.

U.S. Pat. No. 5,663,195 discloses a method of inhibiting bone resorption by administration of a selective cyclooxygenase-2 inhibitor. This method halts or retards loss of bone, promotes bone repair and aids in prevention of fractures.

U.S. Pat. Nos. 5,763,416 and 5,942,496 disclose methods for the transfer of osteotropic genes (genes for parathyroid hormone, BMP's, growth factors, growth factor receptors, cytokines and chemotactic factors) into bone cells for treatment of bone-related diseases and defects.

U.S. Pat. No. 5,962,427 discloses a method for specific targeting and DNA transfer of a therapeutic gene into mammalian repair cells. The modified repair cells proliferate and populate a wound site while expressing the therapeutic gene.

Dr. Brunilda Nazario reports on a drug, FORTEO (teriparatide), derived from parathyroid hormone, which is useful in the treatment of osteoporosis (accessed from the WebMD website on Dec. 23, 2003). Teriparatide is a bone formation agent that promotes bone growth by increasing the number and activity of bone-forming cells (osteoblasts).

A substantial amount of research has been conducted to elucidate methods for improved healing of skeletal defects; resulting in, for example, immobilization devices and bone grafts.

Many devices have been constructed for application to the area of a bone fracture in order to immobilize, facilitate and support healing and prevent deformities, such as the devices disclosed in U.S. Pat. Nos. 5,853,380; 5,941,877; and U.S. Patent Application Nos. 2003/0181979 and 2003/0099630.

Another conventional approach in treating lordosis is taught by Liu et al, U.S. Pat. No. 6,746,484, which discloses a spinal cage packed with bone growth material to be implanted in the intervertebral disk space to provide support and proper spinal curvature during the process of fusion of the adjacent vertebrae.

Methods involving the replacement of damaged bone tissue with a bone graft are more common. A bone graft can be prepared from autograft tissue(bone tissue is obtained from a site other than the damaged bone area in the same individual requiring the graft), allograft tissue (bone tissue is obtained from a donor) or can be constructed from artificial materials.

Use of allograft tissue avoids donor site complications in the tissue recipient, additionally such tissue can be obtained in large quantities. However, many disadvantages arise when using allograft tissue, including, expense, possible disease transmission and detrimental host response. Allan E. Gross (Orthopedics 26(9) :927-928 September 2003) discusses use of allograft tissue in reconstructive surgery in the lower extremities.

Bauer et al. (Orthopedics 26(9) :925-926 September 2003) present a general discussion of four categories of available bone graft substitutes; hydroxyapatite products, soluble calcium-based blocks/granules, injectable cements and osteoinductive materials.

Generally derived from sea coral, hydroxyapatite products are osteoinductive and possess compressive strength. These products can be brittle, difficult to prepare and slow to resorb once implanted. Examples of the use of hydroxyapatite products in bone tissue repair can be found, for example, in U.S. Pat. Nos. 6,585,992; 6,290,982; 6,206,957; 5,069,905 and 5,015,677.

Soluble calcium-based blocks/granules facilitate the mineral deposition which is necessary for bone remodeling. Lee Beadling (Orthopedics Today, page 43, November 2003) discloses an injectable calcium sulfate graft having improved compressive strength and resorption properties.

Yu et al. (U.S. Application No. 2002/0169210, published on Nov. 14, 2002) disclose a method for treating and preventing fractures with administration of calcium L-threonate. Calcium L-threonate was found to promote proliferation, differentiation and mineralization of osteoblasts and also found to promote expression of collagenI mRNA in osteoblasts. Yu et al. disclose that treatment with calcium L-threonate facilitated bone fracture healing and increased bone density and mechanical performance thus preventing bone fracture. In the method of Yu et al. calcium L-threonate was taken systemically (orally or parentally) and was not applied directly to the desired location in specific bones as in the method of the instant invention.

Cements which are capable of injection at fracture sites or sites of implantation of prosthetic devices act as bonding material for improving fracture healing and for securing prosthetic devices. Injectable cements vary in useful properties; for example; calcium phosphate is osteoconductive, has compressive strength, slow resorption, and is weak in tensile strength and shear while silica based cements are strong but weakly osteoinductive. There are many cements and devices for their use known in the art, for example, the isovolumic mixing and injection device disclosed by James Marino in U.S. Pat. No. 6,406,175.

Demineralized human bone tissue, termed bone matrix when mixed with a carrier such as glycerol, is powerfully osteoinductive and naturally contains growth factors which aid in healing bone, such as bone morphogenetic proteins (BMP's). BMP's were first identified from demineralized bone and were found to function as signal transducing proteins in the processes of skeletal development and bone formation. Currently, BMP's are under clinical investigation as potential facilitators of bone and cartilage repair.

In contrast to the instant invention, the prior art does not disclose the use of structural implants containing a reservoir of BMP's for prevention of further fractures in a fractured bone, an unfractured bone or in a bone susceptible to fracture before fracture occurs. The instant devices are the first to combine administration of BMP's to unfractured bone for the prevention of fractures with a structural support to increase rigidity.

### SUMMARY OF THE INVENTION

The instant invention provides a method and device useful for reducing susceptibility to vertebral compression fractures, particularly in osteoporotic vertebrae. The method achieves enhanced bone growth in areas specifically affected by osteopenia, thus increasing bone density in these affected areas and reducing susceptibility of the thinning bones to fracture. The method is particularly suited to the treatment of vertebral bodies and can minimize the risk for additional vertebral compression fractures (VCF) after initial VCF occurs.

The method generally is accomplished through carrying out three basic steps; formulating a bone bone growth enhancing agent or mixture, providing a structural implant carrying the mixture, administering the implant to the core of a vertebral body and distributing the agent or mixture into the regions of the cancellous medullary cavity most at risk for vertebral body fracture. The region adjacent the end plate of the vertebral body is generally the preferred site of implantation. The method may be practiced separately or practiced in consort with other procedures, non-limiting examples of which include, disk arthroplasty, dynamic stabilization operations, vertebroplasty, kyphoplasty and during surgical repair of existing fractures in order to prevent additional collapse of cortical/cancellous bone within the already fractured vertebrae.

The first step involves formulating an agent or mixture including bone matrix and/or at least one bone growth enhancing agent. A mixture may include bone matrix alone, a bone growth enhancing agent alone or combinations of bone matrix and bone growth enhancing agents. Bone matrix may be combined with a single bone growth enhancing agent or with multiple bone growth enhancing agents. Any material which enhances bone growth is contemplated for use in the solution of the instant invention; illustrative, albeit non-limiting examples of such materials are bone morphogenetic proteins (BMP's), cytokines, hormones, bone matrix, gene therapy agents, electrical stimulation agents and growth factors.

The instant invention also provides means for administration of the mixture. The means for administration is a device constructed and arranged for controlled deposition of the solution into the medullary cavity and onto the interior cancellous surface of the vertebral body. The form of the device may be illustrated as a cylinder, block or sphere. The device may or may not have hollow voids. Since the rate of bone thinning varies for each individual and even varies at different rates in separate areas of the same individual, one insert design may not be ideally suited to every situation.

The second step of the method involves administration of the mixture into the medullary cavity of the vertebral body by use of the device inserted into it's intramedullary space through an aperture. The device can be introduced into the aperture percutaneously, either transpedicular, lateral extra pedicular or posterolateral or anteriorly or latterally, as an alternative application.

The third step of the method involves distribution of the mixture into the region of the medullary cavity of the vertebral body in a way that allows the mixture contact with the cancellous tissue effective for achieving active bone restoration as a result of controlled deposition of the mixture, while instantly reconstituting the loss of structural support caused by creating an aperture into an unfractured bone. Additionally, the mixture will disperse, by flowing through the cancellous bone channels, to contact the cancellous portion of the vertebral body and the subchrondral cortical-cancellous bone defined as the vertebral end plate.

The mixture may be administered in a single dose, in multiple doses over periods of time or may be formulated for controlled release. To administer multiple doses over periods of time, sequential access to, and deposition within, of the mixture will occur.

Although the method and device of the instant invention are exemplified by administration to an unfractured bone which has been determined to be at risk for fracture (at-risk bone), they may also be administered to a fractured bone to improve healing by enhancing growth of the newly formed bone or to prevent additional subsequent fractures of newly healed bone, or bone not yet fractured. The instant invention is contemplated for use with any bone-related disease and/or defect which may involve thinning, weakened and/or damaged bones; illustrative, albeit non-limiting situations are, osteoporosis, after a traumatic injury to a limb with resultant osteopenia from disuse or immobilization, corticosteroid regimens, osteogenesis imperfecta, complications with prosthetic devices and bone damage due to radiation treatments and bone damage due to tumor invasion.

Accordingly, it is an objective of the instant invention to provide a device for reducing susceptibility to fractures in bones.

It is yet another objective of the instant invention to provide a device constructed and arranged for controlled deposition of a solution into the medullary cavity and onto the interior surface of a bone.

It is yet another objective of the instant invention to provide a device for reducing susceptibility to fractures in vertebral bodies by providing internal support to weakened bones.

It is still another objective of the instant invention to provide a device constructed and arranged for controlled deposition of a solution into the medullary cavity and onto the interior cancellous surface of a vertebral body.

It is a further objective of the instant invention to provide a device that will provide instantaneous structural support to the medullary cavity aperture utilized to implant the device.

It is a yet further objective of the instant invention to provide a device conductive to direct current electricity for enhancing bone growth.

Other objectives and advantages of the instant invention will become apparent from the following description taken in conjunction with the accompanying drawing(s) wherein are set forth, by way of illustration and example, certain embodiments of the instant invention. The drawing(s) constitute a part of this specification and include exemplary embodiments of the present invention and illustrate various objects and features thereof.

### BRIEF DESCRIPTION OF THE FIGURES

FIG. 1 is a perspective of the implant of this invention;

FIG. 2 is a longitudinal cross section along line 2-2 of FIG. 1;

FIG. 3 is a cross section of another embodiment of the implant of this invention; and

FIG. 4 is a cross section showing the drill pin.

### ABBREVIATIONS AND DEFINITIONS

The following list defines terms, phrases and abbreviations used throughout the instant specification. Although the terms, phrases and abbreviations are listed in the singular tense the definitions are intended to encompass all grammatical forms.

As used herein, the term “vertebral body” refers to the rounded anterior segment of a skeletal vertebra.

As used herein, the abbreviation “VCF” refers to a vertebral compression fracture.

As used herein, the term “kyphosis” refers to a condition wherein the spine falls forward and is shortened in length, usually due to vertebral compression fractures.

As used herein, the term “osteoplasty” refers to any surgical procedure or process by which total or partial loss of bone is remedied.

As used herein, the term “vertebroplasty” refers to a surgical procedure wherein a bone cement is injected into the center of a fractured vertebrae through a tube inserted into a small aperture in the tissue. The bone cement stabilizes the fracture, which relieves pain and prevents further collapse of the vertebra.

As used herein, the term “kyphoplasty” refers to a surgical procedure similar to vertebroplasty which additionally includes partial restoration of height and creation of bone by inflation of a balloon within the medullary cavity prior to injection of the cement.

As used herein, the term “bone mineral density test” refers to an X-Ray process wherein the amount of calcium in bones is determined and bone strength is ascertained. The most common areas for application of bone mineral density testing are the hip and the spine. This test is used most often to detect osteoporosis.

As used herein, the abbreviation “DEXA” refers to dual energy X-ray absorptiometry; a type of bone mineral density test wherein two X-ray beams are applied to the bone and the amounts of each X-ray beam blocked by bone and tissues are compared to estimate bone density.

As used herein, the abbreviation “P-DEXA” refers to a modification of the DEXA test wherein bone density in peripheral bone areas such as the wrist is measured.

As used herein, the abbreviation “DPA” refers to dual photon absorptiometry; a type of bone mineral density test similar in principle to the DEXA test; but instead uses a radioactive material to produce photons which are applied to bone (in place of X-ray beams).

As used herein, the term “ultrasound” refers to a type of bone mineral density test which utilizes sound waves reflected from bones in peripheral areas of the body to measure bone density.

As used herein, the phrase “at-risk bone” refers to a bone which has been determined to be at risk for fracture; due to identified fragility, presence adjacent to a fractured bone or any other identifiable risk factors for fracture.

As used herein, the term “bone matrix” refers to human bone tissue which has been demineralized and combined with a carrier material such as glycerol or starch. Bone matrix naturally contains bone growth enhancing agents.

As used herein, the term “bone growth enhancing agent” refers to any injectable biological and/or synthetic molecule, cell, gene or material which facilitates and/or increases the rate of bone growth or favorably improves the balance of bone resorption to bone deposition. A bone growth enhancing agent can also be referred to as a bone growth accelerator.

As used herein, the term “controlled deposition” refers to the ability of the device for distribution of the bone growth enhancing agent to control release of the solution to the interior surface area of the bone. The physical and biological properties the device combine to control the precise location and rate of deposition of the solution in the medullary cavity and to prevent any biologic adverse impact on bone or soft tissue structures away from the intended medullary cavity and vertebral end plate.

As used herein, the abbreviation “BMP” refers to bone morphogenetic protein. “rhBMP” refers to recombinant, human bone morphogenetic protein. BMP's are signal transducting proteins of the transforming growth factor-beta superfamily which function in skeletal development and bone formation. BMP's were first identified in demineralized bone.

As used herein, the phrase “naturally contains” refers to any substance or material which occurs in nature or is naturally present in a living or previously living organism, for example, bone matrix as obtained from a human tissue donor naturally contains BMP's but does not naturally contain recombinant BMP's or other such recombinant proteins.

The terms “surgical wound” and “incision” are used interchangeably herein.

### DETAILED DESCRIPTION OF THE INVENTION

The mixture, as formulated according to the instant invention, may include bone matrix alone, a bone growth enhancing agent alone or combinations of bone matrix and bone growth enhancing agents. Any bone cement known in the art can also be added to the mixture or can replace bone matrix in the mixture. Bone matrix may be combined with a single bone growth enhancing agent or with multiple bone growth enhancing agents. As bone matrix is derived from human bone tissue, it naturally contains bone growth enhancing agents. The addition of at least one bone growth enhancing agent to the bone matrix mixture may increase the effectiveness of the treatment. Additional bone growth enhancing agents can be obtained from any tissue source or can be recombinantly produced. Any natural and/or synthetic material which enhances bone growth is contemplated for use in the solution of the instant invention, illustrative, albeit non-limiting examples of such materials are BMP's, cytokines, hormones, gene therapy agents, DC electrical stimulation, and growth factors. Illustrative, albeit non-limiting examples of BMP's are any of the fourteen types of human BMP's (BMP's 1-14). Cytokines are polypeptides transiently produced by many different types of cells and function as intercellular messengers, usually by binding to cell surface receptors. Illustrative, albeit non-limiting examples of cytokines are interferons, tumor necrosis factors, lymphokines, colony-stimulating factors and erythropoietin. Hormones are also organic intercellular messengers. Illustrative, albeit non-limiting examples of hormones are steroid hormones, prostaglandins, peptide H, adrenalin and thyroxin. Growth factors are mitogenic polypeptides functioning in intercellular signaling. Illustrative, albeit non-limiting examples of growth factors are platelet derived growth factor, transforming growth factors and epidermal growth factor. A radioopaque material can also be added (to the solution) in order to facilitate visualization of the administration and distribution of the device. The volume and concentration of solution will be formulated on a per case basis since volume and concentration of the solution depends on the volume of the bone to be treated, as well as the biological and physical properties of the solution. The quality (degree of thinning) of the bone to be treated determines the type of administration, for example, a single dose of solution, multiple doses of solution over a period of time, or a solution formulated for controlled release after administration, e.g. formulated within a carrier of limited solubility, encapsulated within a slowly degrading device, or the like.

The device for administration is a device constructed and arranged for controlled deposition of the solution into the medullary cavity and onto the interior cancellous surface of the vertebral body including transmission of the solution to the end plate. Additionally, since the rate of bone thinning varies for each individual and even varies at different rates in separate areas of the same individual, one design of the device may not be ideally suited to every situation. The degree of thinning is assessed by bone mineral density testing. Illustrative, albeit non-limiting examples of bone density testing are DEXA, P-DEXA, DPA and ultrasound.

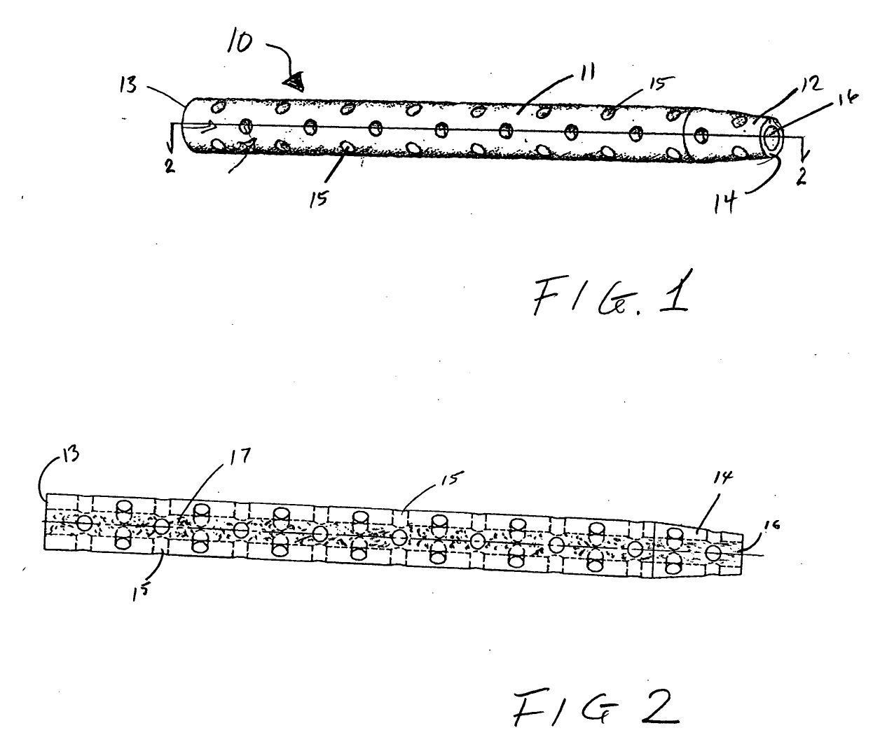

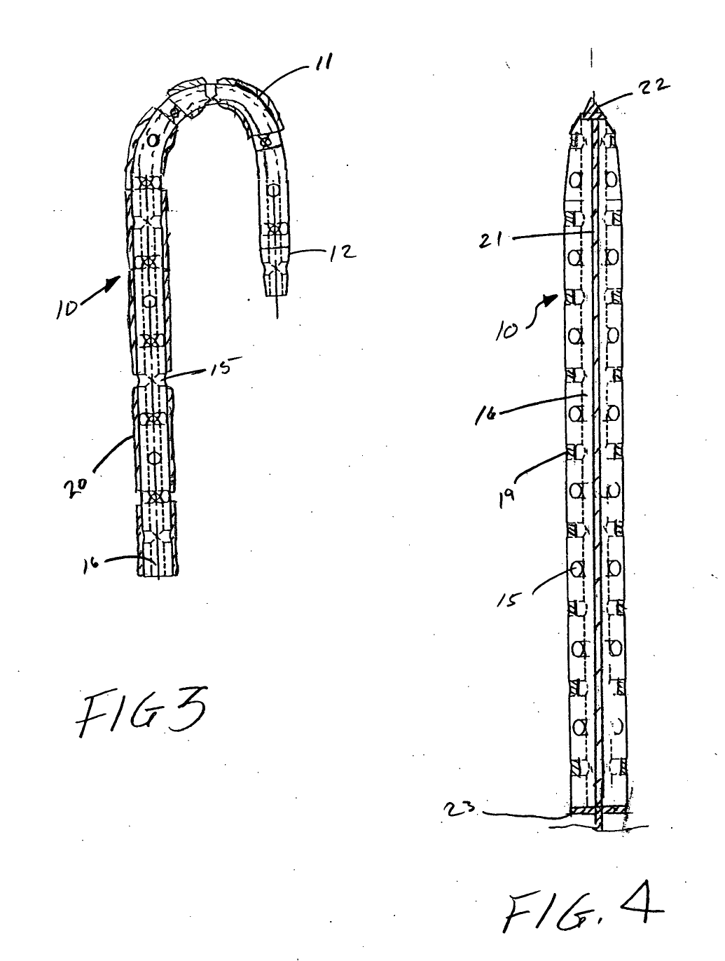

The implant 10 of the instant invention are particularly suited to the treatment of vertebral bodies although the device may be implanted in other skeletal components. As shown in FIG. 1 and FIG. 2, the implant 10 is formed as an elongated cylindrical body 11 having a leading end 12 and a trailing end 13. A series of perforations 15 are spaced throughout the length of the body in a repeating or random pattern. The perforations 15 penetrate the cylinder wall 14 and communicate with the cylinder bore 16. The bore 16 may be filled with a bone growth mixture 17 of a particular formulation depending on the specific case. The implant 10 may be pre-packaged with different materials allowing the surgeon to pick a certain implant for a particular case. Or the implant may be supplied empty and the mixture may be added to the implant before the procedure. Or the implant may be inserted into the bone empty and then filled, in situ. The perforations may be temporarily closed with a soluble material 19, if the mixture is liquified.

The implant 10, per se, may be made of the bone growth mixture compressed, or otherwise treated, to become a self sustaining form with or without a different mixture in the bore 16. The bone growth mixture may be mixed with other bio-absorbable ingredients to add temporary rigidity and internal support in the bone. These absorable materials contribute to the instantaneous internal support of the bone and form a temporary implant.

The cylindrical body 11 may be made of non-absorbable materials, such as bone cement or other bio-compatible materials including metals, polymers, carbon fibers, and the like, containing the bone growth mixture in the bore and, if desired as an exterior coating 20. These non-absorbable materials contribute to the internal support of the bone and form a permanent implant. The body 11, if made of radiolucent or non-metallic material, will be impregnated with at least two radiopaque markers for peri-operative image guidance and post-procedure monitoring of the device location. A metallic bead, conventional in the industry, will be utilized, as an illustrative, albeit non-limiting example.

The metals used for the cylindrical body include heat sensitive Nitinol pre-formed to assume a circular or spiral shape upon exposure to body temperature. The implant 10 would then deform, in situ, to generally conform to the interior of the bone. The metals can also be conductive to an electrical charge whereby when exposed to an electrical field the bone can be stimulated for increased growth.

As shown in FIG. 4 the implant 10 may have a shaft 21 extending through the bore 16. The shaft 21 can be in the form of a “Steinman pin” with a bone drilling tip 22. The leading end of the cylindrical body 11 is tapered to a smaller diameter to present a smooth transition to the tip 22. The trailing end of the cylindrical body 11 is held in place by a flange 23 slidably mounted on the shaft 21 of the pin. Once the cancellous shell of the bone has been breached by the drill, the cylindrical body 11 may be advanced into the cancellous bone by pressure on the flange 23. The introducing pin may then be removed after implanting. The bore of the implant may then be filled with the selected bone growth material by a cannula. Alternatively, the bore of the implant may be filled with bone growth material around the shaft of the pin and the implant and pin may remain in the bone as support.

After preparation of the solution and the device, an incision is made in the tissue (including the bone) in order to form an intramedullary aperture for insertion of the implant. The incision must be of a width sufficient for insertion and maneuverability of the device within the medullary cavity of a bone, such as a vertebral body. Bi-planar fluoroscopic or image-guided systems are used to guide the introduction of the implant into the vertebral body.

After insertion of the implant, the solution is distributed into the interior cavity of a vertebral body and diffuses in a way that allows the solution contact with the cortical and cancellous tissue effective for achieving active bone restoration. Distribution may be carried out by spraying or injecting the solution. Controlled release by leaching of the bonded solution out of the implant may also occur. The distribution of solution should always be carried out by “controlled deposition”. Controlling the deposition of the solution is necessary to assure that precise amounts of solution are distributed in a manner which avoids unintentional fracture, excessive mechanical disruption or extrusion of the solution into extraosseus locations.

The following protocol is designed to be carried out to treat an individual with osteoporosis involving the thoracic and lumbar vertebrae. This protocol would be generally implemented in patients undergoing vertebroplasty, kyphoplasty, osteoplasty or other methods of vertebral augmentation for a vertebral body fracture or fractures. This protocol is designed for treatment of “at-risk” vertebral bodies, those vertebral bodies which are not fractured but are at risk for fracture due to deformity caused by previous fracture to other vertebral bodies and/or the degree of osteoporosis in the non-fractured vertebrae. The procedure may be utilized in patients without prior fracture, poorly responsive to alternative pharmacologic agents, and with bone density testing which reveals severe risk for fracture.
1. One would first determine the volume of the vertebral body by mathematical calculation of the volume of the cylinder portion combined with a modifier based upon bone density as determined by bone density testing. This calculation allows for the volume and formulation of bone growth enhancing solution to be determined;
2. One would then prepare the solution in the pre-determined amount and formulation, adding additional bone growth enhancing agents if desired;
3. One would then select the desired implant design, size, length, diameter and insert (s) which best suits the needs of the individual patient to be treated and load the selected insert with the formulated bone growth enhancing solution;
4. One would then prepare an incision in the tissue (including the bone), after adequate anesthesia, which is of significant width to allow insertion and maneuverability of the implant in the medullary cavity of the vertebral body to be treated. Via either the posterior, percutaneous, minimally-invasive transpedicular extrapedicular or the percutaneous posterolateral approach, one would then pass the implant having an insert (Steinman pin) with a modified sharpened end into the vertebral body to prepare a clear pathway for deposition of the solution, alternatively, and less common, the anterior or lateral approach may be utilized;
5. If the implant is empty, one would then withdraw the pin having the modified sharpened end and next engage a cannula to administer the bone growth enhancing solution by either injection or spray;
6. The implant would then distribute the bone growth enhancing solution by controlled deposition within the desired region of the interior cavity of the vertebral body; and
7. One would then close the incision to complete the procedure.
   The post-procedure follow-up of the individual patient would include X-rays and/or bone density tests over a period of time in order to track the bone restoration in the treated vertebral body.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification.

What is claimed is:

1. A bone implant for increasing bone density and providing internal support comprising a rigid elongated body adapted to be inserted into the interior of a bone combined with a bone growth mixture whereby said rigid elongated body prevents collapse of the bone and said bone growth mixture increases bone density.

2. A bone implant for increasing bone density and providing internal support of claim 1 comprising said rigid body formed as a cylinder having a bore, said cylinder having a series of perforations communicating with said bore, said bone growth mixture in said bore.

3. A bone implant for increasing bone density and providing internal support of claim 1 comprising said rigid elongated body formed in a cylinder, an elongated pin disposed in said cylinder, said pin having a leading end and a trailing end, said pin reinforcing said cylinder.

4. A bone implant for increasing bone density and providing internal support of claim 2 comprising said cylinder formed of a heat sensitive material whereby said cylinder deforms into a preformed shape when exposed to body temperature in a bone.

5. A bone implant for increasing bone density and providing internal support of claim 3 comprising said leading end tapered to a point, said point adapted to drill an aperture into a bone whereby said elongated body and said pin are adapted to be inserted through the aperture into a bone.

6. A bone implant for increasing bone density and providing internal support of claim 3 comprising said cylinder being electrically conductive whereby said implant is adapted to increase bone growth when electrical energy applied to said cylinder.

7. A bone implant for increasing bone density and providing internal support of claim 2 comprising an elongated pin disposed in said cylinder, said pin having a leading end and a trailing end, said pin reinforcing said cylinder, a plunger on said trailing end of said pin, said plunger contacting said cylinder whereby said elongated body is fixed on said pin.

8. A bone implant for increasing bone density and providing internal support of claim 7 comprising said leading end tapered to a point, said point adapted to drill an aperture into a bone whereby said elongated body and said pin are adapted to be inserted through the aperture into a bone.

9. A bone implant for increasing bone density and providing internal support of claim 8 comprising said cylinder having a tapered portion of lesser diameter, said tapered portion contacting said tip, said tip being of lesser diameter than said bore, said plunger slidably attached to said trailing end of said pin, said plunger adapted to slide toward said tip forcing separation of said cylinder and said pin whereby said pin is adapted,to be withdrawn from a bone.
